(19) United States
(12) Patent Application Publication
(10) Pub. No.: US 2019/0282406 A1
(43) Pub. Date: Sep. 19, 2019

(54) STRETCHABLE BANDAGE TAPE AND METHOD OF USE

(71) Applicant: GA INTERNATIONAL INC., Laval (CA)

(72) Inventor: Gourgen AMBARTSOUMIAN, LAVAL (CA)

(21) Appl. No.: 16/301,645

(22) PCT Filed: May 18, 2017
(86) PCT No.: PCT/CA2017/050603

§ 371 Date: Nov. 14, 2018

Related U.S. Application Data
(60) Provisional application No. 62/338,107, filed on May 18, 2016.

(51) Int. Cl. A61F 13/02; A61L 15/58

(52) U.S. Cl. CPC A61F 13/0269; A61L 15/58

(57) ABSTRACT

A bandage tape comprises a strip body having a first surface and a second surface, the first surface and the second surface being made of one of a silicone and a silicone rubber. A thickness between the first surface and the second surface ranges between 0.5 mil-120.0 mil, the strip body configured for the first surface at a first end to self-adhere to the second surface at a second end by overlaying in a silicone-to-silicone bond. The strip body is configured for the second surface at the first end to self-adhere to the first surface at the second end by overlaying in a silicone-to-silicone bond. A use of a combination of a bandage tape, and dressing for bandaging an anatomical body part is provided, as well as a method for bandaging an anatomical body part with the bandage tape.

### CROSS-REFERENCE TO RELATED APPLICATION

The present application claims priority on U.S. Patent Application Ser. No. 62/338,107, filed on May 18, 2016 and incorporated herein by reference.

### FIELD OF THE INVENTION

The present application relates to a bandage tape that can be wrapped around a body part of a human or non-human being, for medical, veterinary or related fields of use, wherein it can be used as a bandage or used for supporting a bandage. Furthermore, the present application relates to a bandage tape used for wound treating, stoppage of bleeding, drug delivery, as a medical device, orthopaedic, surgical, device attachment to a body, clinical sample taking procedures, injury relief and protection, among possible uses.

### BACKGROUND OF THE ART

The use of bandages in wound and injury treatment is routine in medical and veterinary practice. There are various types of bandages and wound-care dressings applied to wounds and injury areas of the body. Such bandages include for example gauze, cotton and other types of cloth, tissue or synthetic materials and dressings. Use of bandages is common in various types of medical, surgical and in outpatient practices which include as an example the following types: dry dressings, wet-to-dry dressings, chemical-impregnated dressings, foam dressings, alginate dressings, hydrofiber dressings, transparent film dressings, hydrogel dressings, hydrocolloid dressings, wound-closure materials, binders, among other known types of dressings and bandages.

Bandages and dressings are commonly available in pharmacies and other stores for personal use. Bandages are routinely used by hospitals, clinics and other medical and veterinary facilities. Commonly known bandages may contain a self-adhesive part such as Band-Aid™ adhesive bandages. Other types of bandages may not have any adhesive and need to be fixed to a body using a medical adhesive tape such as the 3M® Tape or similar. For example, it is a daily routine in clinical laboratories to take blood samples wherein a needle is inserted into a blood vessel, to withdraw some blood. Subsequently, a cotton pad or other type of bandage is fixed on the needle penetration site to stop the bleeding. The most common way of fixing the bandage is using an adhesive tape. For intravenous injections and blood-withdrawing from a patient on frequent intervals often the intravenous (IV) needle is inserted in the blood vessel and fixed there for extended periods (e.g., hours, days) using an adhesive tape. In some cases, the adhesive tape is wrapped around the bandage and often makes contact with a skin. In other cases the adhesive tape is just fixed to the skin without wrapping. Some of the drawbacks of using such tapes may be that i) the adhesive sticks to the skin and/or hair and upon removal causes pain and discomfort to a patient; ii) the adhesive may be sensitive to water, soap, shampoo and detergents and upon washing or showering the tape may detach and expose the bandage to water and increases the chance for wound contamination; iii) individuals may be allergic to adhesives which may cause irritation or reaction from a patient; iv) adhesive tapes are not fully flexible and may detach when body parts move especially near joints; v) when the skin is wet with a disinfecting solution or any other solution, such as water, cream or ointment, the adhesive tape may not stick and may come off easily.

Various types of injuries may cause bleeding. Stopping the blood loss is an essential step in preventing further deterioration of the medical condition. In some instances, the outcome may be dependent on how fast the bleeding was stopped. Currently, a common recommendation for stopping bleeding is to apply pressure on the wound area, for instance by using a tourniquet. The difficulty is that in emergency situations it takes one person to provide first aid to the injured, for example by applying pressure to stop the bleeding, but if that person leaves to call for emergency services or find other help the bleeding may resume. In some situations, when make-shift tourniquets are made or used, they need to be fixed with enough pressure to stay in place and prevent the bleeding. Most materials available for first aid cannot seal the wound and apply the pressure simultaneously which would assist in stopping the bleeding without having a second person to press and hold the bleeding vessels.

### SUMMARY

It is therefore an aim of the present disclosure to provide a silicone or a silicone-bandage tape that addresses issues related to the prior art.

It is a further aim of the present disclosure that the bandage tape serve as a bandage in contacting directly the skin and/or injured part of a body.

Therefore, in accordance with a first embodiment of the present disclosure, there is provided a bandage tape comprising a strip body having a first surface and a second surface, the first surface and the second surface being made of one of a silicone and a silicone rubber, a thickness between the first surface and the second surface ranging between 0.5 mil-120.0 mil, the strip body configured for the first surface at a first end to self-adhere to the second surface at a second end by overlaying in a silicone-to-silicone bond, the strip body further configured for the second surface at the first end to self-adhere to the first surface at the second end by overlaying in a silicone-to-silicone bond.

Further in accordance with the first embodiment, the strip body is in an instance a monolithic body of silicone or silicon rubber.

Still further in accordance with the first embodiment, printing is in an instance on at least one of the first surface and of the second surface.

Still further in accordance with the first embodiment, the strip body is in an instance non-continuous and divided into a section sized for a contemplated used.

Still further in accordance with the first embodiment, the non-continuous strip body is in an instance in an individual package in a sterile condition.

Still further in accordance with the first embodiment, the strip body is in an instance in a roll, and further comprising a backing layer placed between the first surface and the second surface of the bandage tape in the roll.

Still further in accordance with the first embodiment, pre-perforated tear lines are in an instance transversely positioned and spaced apart along the strip body.

Still further in accordance with the first embodiment, the strip body has in an instance a width ranging between 0.25″ and 6.0″.

In accordance with a second embodiment of the present disclosure, there is provided a method for bandaging an anatomical body part comprising: obtaining a bandage tape comprising a strip body having a first surface and a second surface, the first surface and the second surface being made of one of a silicone and a silicone rubber, a thickness between the first surface and the second surface ranging between 0.5 mil-120.0 mil; placing a first end of the strip body against the anatomical body part to be bandaged; wrapping and stretching the strip body around the anatomical body part; and forming a silicone-to-silicone bond by overlaying the second surface at a second end onto the first surface at the first end, whereby the strip body self-adheres to itself while stretched around the anatomical body part.

Further in accordance with the second embodiment, forming the silicone-to-silicone bond by overlaying the second surface at a second end onto the first surface at the first end comprises in an instance forming the silicone-to-silicone bond while the strip body is over a wound on the anatomical body part, whereby the strip body self-adheres to itself while stretched to cover said wound of the anatomical body part.

Still further in accordance with the second embodiment, forming the silicone-to-silicone bond while the strip body is over a wound on the anatomical body part comprises in an instance forming the silicone-to-silicone bond while the strip body is directly in contact with said wound.

Still further in accordance with the second embodiment, a dressing is placed on the wound, and forming the silicone-to-silicone bond includes in an instance forming the silicone-to-silicone bond such that the dressing is between the wound and the strip body.

Still further in accordance with the second embodiment, forming the silicone-to-silicone bond such that the dressing is between the wound and the strip body comprises in an instance completely concealing the dressing between the strip body and the anatomical body part.

Still further in accordance with the second embodiment, at least one of a disinfectant and ointment is in an instance applied to the anatomical body part prior to bandaging the anatomical body part, whereby the bandage tape is directly in contact with the at least one of a disinfectant and ointment.

Still further in accordance with the second embodiment, medication is in an instance applied to the anatomical body part prior to bandaging the anatomical body part, whereby the medication is held captive and in contact with the anatomical body part.

Still further in accordance with the second embodiment, stretching the strip body around the anatomical body part comprises in an instance stretching the strip body by at least 50%.

Still further in accordance with the second embodiment, obtaining a bandage tape comprises in an instance unrolling a length of the bandage tape from a roll, sectioning the bandage tape to define the strip body, and removing a backing layer from the strip body.

Still further in accordance with the second embodiment, obtaining a bandage tape comprises in an instance removing the strip body from an individual package.

Still further in accordance with the second embodiment, a wireless device is placed in an instance between the strip body and the anatomical body part, whereby the wireless device is maintained captive against the anatomical body part.

Still further in accordance with the second embodiment, a medical device interacts in an instance with the anatomical body part between the strip body and the anatomical body part, whereby the medical device is maintained captive against the anatomical body part.

Still further in accordance with the second embodiment, placing a medical device comprises in an instance placing any one of a splint, a cast, a rod, a drug delivery system, a needle, a syringe, a tube, and a catheter.

Still further in accordance with the second embodiment, obtaining a bandage tape comprises in an instance selecting a color of the bandage tape as a function of the contemplated use.

Still further in accordance with the second embodiment, the silicone-to-silicone bond by overlaying the second surface at a second end onto the first surface at the first end comprises in an instance forming a tourniquet to reduce blood flow on the anatomical body part.

In accordance with a third embodiment of the present disclosure, there is provided a use of a combination of a bandage tape, and dressing for bandaging an anatomical body part, the use comprising: a dressing for being applied against a wound on the anatomical body part; and a bandage tape comprising a strip body having a first surface and a second surface, the first surface and the second surface being made of one of a silicone and a silicone rubber, a thickness between the first surface and the second surface ranging between 0.5 mil-120.0 mil, the strip body configured for the first surface at a first end to self-adhere to the second surface at a second end by overlaying in a silicone-to-silicone bond, the strip body further configured for the second surface at the first end to self-adhere to the first surface at the second end by overlaying in a silicone-to-silicone bond.

Further in accordance with the third embodiment, the strip body is in an instance a monolithic body of silicone or silicon rubber.

Still further in accordance with the third embodiment, the strip body has in an instance printing on at least one of the first surface and of the second surface for supporting information related to at least one of a patient, a treatment, a condition, and a medication.

Still further in accordance with the third embodiment, an individual package is in an instance for packaging the bandage tape.

Still further in accordance with the third embodiment, the strip body is in an instance in a roll, and a backing layer is for placing between the first surface and the second surface of the bandage tape in the roll.

Still further in accordance with the third embodiment, pre-perforated tear lines are in an instance transversely positioned and spaced apart along the strip body for sectioning sections of the strip body from the roll.

Still further in accordance with the third embodiment, the strip body has in an instance a width ranging between 0.25″ and 6.0″.

Still further in accordance with the third embodiment, at least one of a disinfectant and ointment is in an instance for treatment of the anatomical body part and for direct contact with the bandage tape.

Still further in accordance with the third embodiment, medication is in an instance for treatment of the anatomical body part and is held captive with the bandage tape.

Still further in accordance with the third embodiment, a wireless device is in an instance between the strip body and the anatomical body part, whereby the wireless device is maintained captive against the anatomical body part.

Still further in accordance with the third embodiment, a medical device interacts in an instance with the anatomical body part between the strip body and the anatomical body part, the medical device being maintained captive against the anatomical body part by the bandage tape.

Still further in accordance with the third embodiment, the medical device is in an instance any one of a splint, a cast, a rod, a drug delivery system, a needle, a syringe, a tube, and a catheter.

Still further in accordance with the third embodiment, the bandage tape has in an instance a color selected from other colors for being identified as a function of the contemplated use.

Still further in accordance with the third embodiment, the bandage tape is in an instance for forming a tourniquet reducing blood flow in the anatomical body part.

Still further in accordance with the third embodiment, the bandage tape is in an instance for restraining movement of the anatomical body part relative to another anatomical body part or to an object.

Still further in accordance with the third embodiment, at least one of wire, a cable, wired electronics, a memory chip, a connector or electrical connection is in an instance connected to an electronic device, and maintained captive against the anatomical body part by the bandage tape.

Yet another aim of the present disclosure is that the bandage tape may be used as a tool or instrument for stopping a bleeding by applying it to the opening on the body surface from where blood exits or distal to the opening to pressure vessels in cases of external and internal bleeding. The bandage tape may serve as a physical barrier to block the external bleeding from the injured vessel by creating a sealed area at the exit wound and apply a pressure to the injured area to decrease or even stop the blood circulation. Furthermore, the stretchable nature (up to 300% or more) of the tape allows the control of the pressure exerted in a particular case. The greater the stretching is prior to applying the bandage tape, the greater the pressure that will be applied on the area of the application upon bonding the tape to itself. In many cases the injured individual may apply the tape alone without external help.

Yet another aim of the present disclosure is securing a splint, an object in orthopaedic or surgical injury to immobilize injured body part using the bandage tape.

Yet another aim of the present disclosure is to use silicone tape for fixing or securing on a body a device capable of injecting, measuring, recording, monitoring, transmitting, or any other type of device. The bandage tape can be used for fixing or securing on a body any instrument comprising but not limited to a needle, syringe, tubing, container, or any other medical object. For the purpose of this invention the term medical or medicine means also veterinary medicine.

Yet another aim of the present disclosure is to use silicone tape for fixing or securing on a body a device capable of injecting, measuring, recording, monitoring, transmitting, or any other type of device. The bandage tape can be used for fixing or securing on a body any instrument comprising but not limited to a needle, syringe, tubing, container, or any other medical or non-medical object.

Yet another aim of the present disclosure is the method of using the bandage tape with any wireless communication device including but not limited to Radio Frequency Identification Tag (RFID) or a Near Field Communication tag (NFC) or a Bluetooth empowered object or a GPS device or a cellular phone.

Yet another aim of the present disclosure is to use the bandage tape for securing or fixing a drug delivery system or any fluid delivery system.

Yet another aim of the present disclosure is that the bandage tape be dispensable in automatic applicators.

Yet another aim of the present disclosure is to provide a kit comprising the bandage tape and any type of dressing and/or a cream, and/or an ointment, and/or a solution, and/or a medication.

Yet another aim of the present disclosure is providing a kit comprising the bandage tape and a wireless communication tag or device including but not limited to a Radio Frequency Identification Tag (RFID) or a Near Field Communication tag (NFC) or a Bluetooth empowered object.

Yet another aim of the present disclosure is the method of using the bandage tape with any wireless communication device including but not limited to Radio Frequency Identification Tag (RFID) or a Near Field Communication tag (NFC) or a Bluetooth empowered object.

### DESCRIPTION OF THE DRAWINGS

FIG. 1A is a perspective view of a bandage tape in accordance with the present disclosure, in a roll on a backing strip;

FIG. 1B is a perspective view of the bandage tape as being detached from the backing strip;

FIG. 10 is a perspective view of the bandage tape as being brought toward a wound on a finger;

FIG. 10 is a perspective view of the bandage tape having a first end applied to the wound;

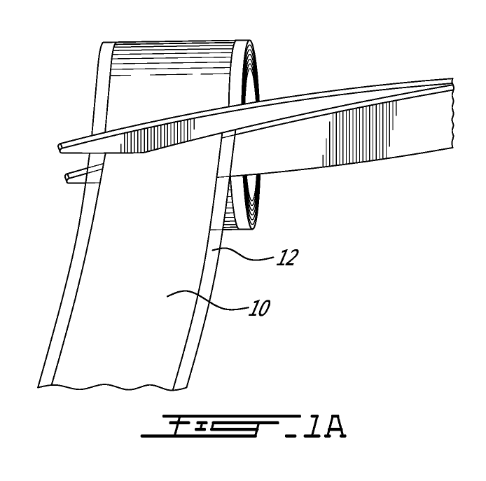

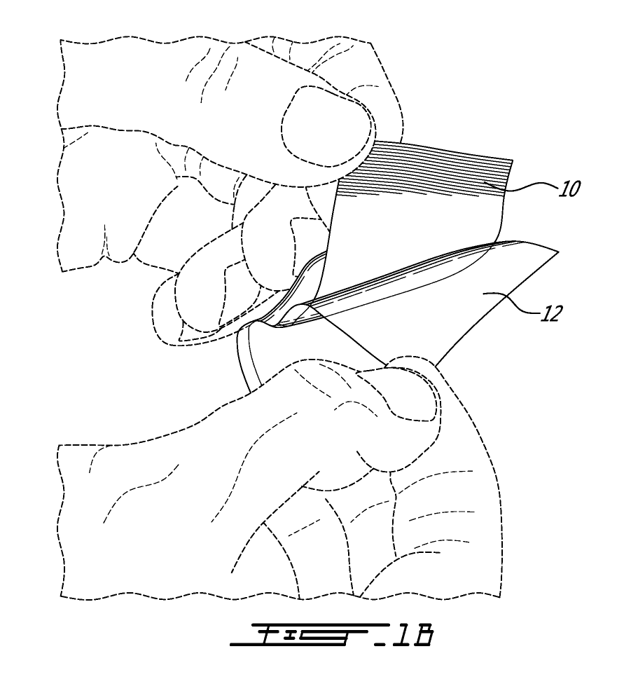

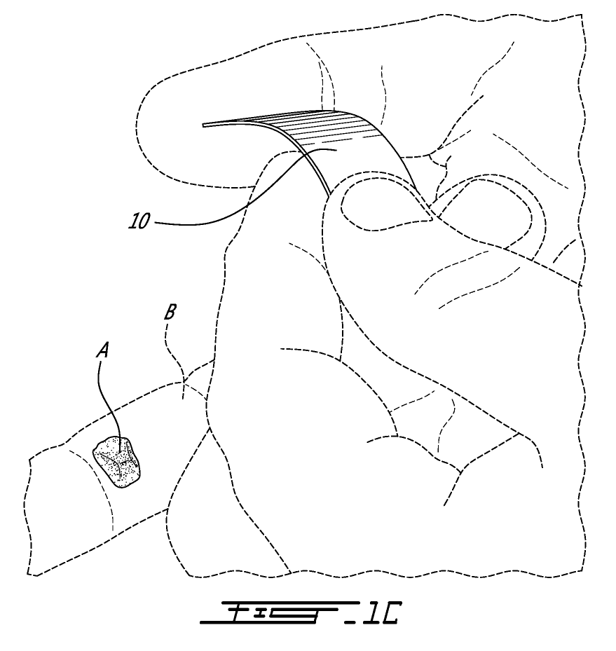

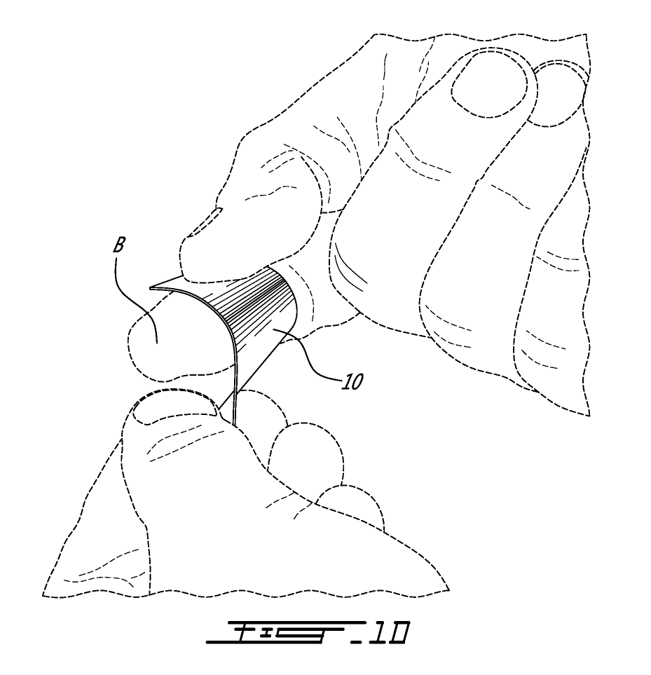

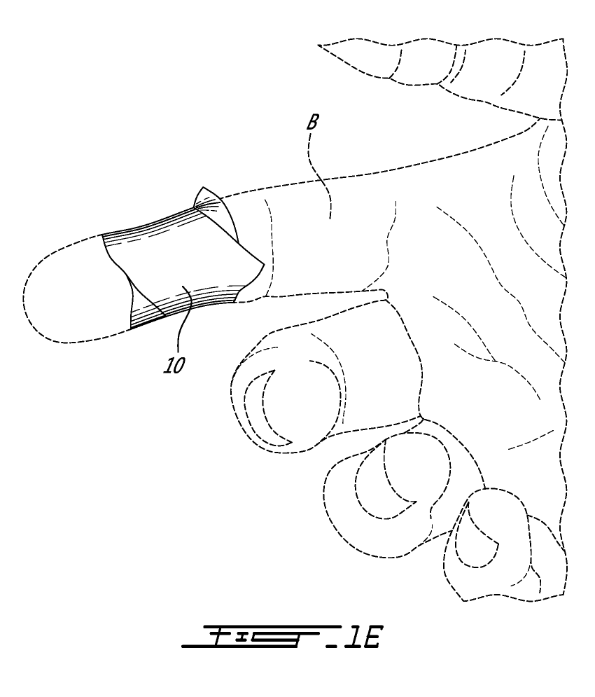

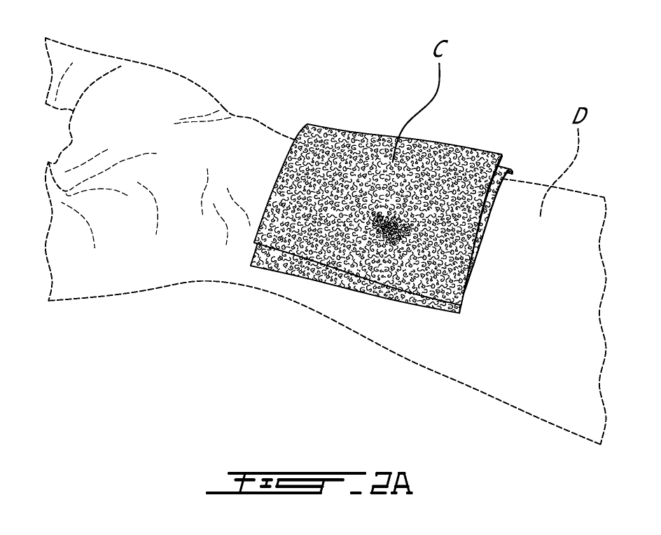

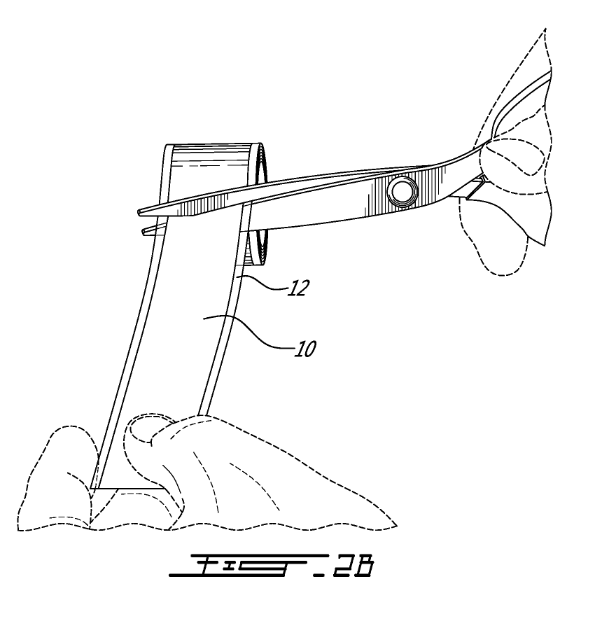

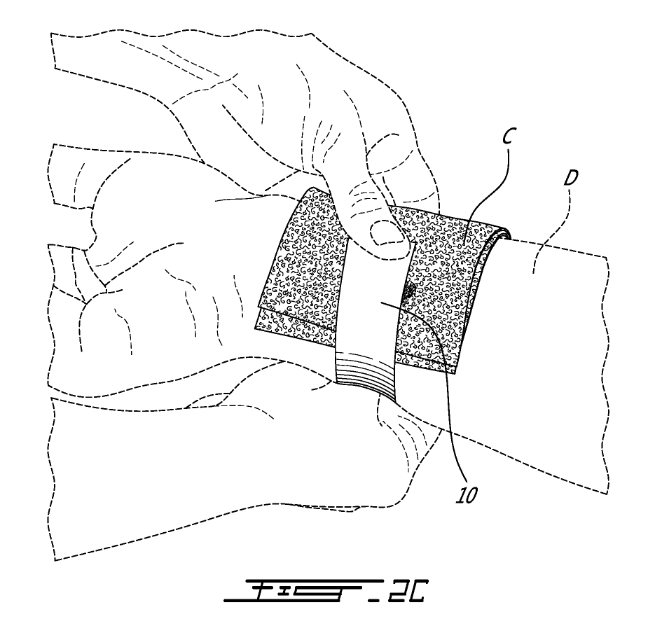

FIG. 1E is a perspective view of the bandage tape attached to the wound on the finger;

FIG. 2A is a perspective view of dressing being applied to a wound;

FIG. 2B is a perspective view of the bandage tape as being cut from the roll;

FIG. 2C is a perspective view of the bandage tape having a first end applied to the dressing;

FIG. 2D is a perspective view of the bandage tape stretched around the forearm;

FIG. 2E is a perspective view of the bandage tape securing the dressing to the forearm:

FIG. 3 is a perspective view of the bandage tape of the present disclosure with pre-perforated tear lines; and

FIG. 4 is a perspective view of a section of the bandage tape of the present disclosure as individually packaged.

### DETAILED DESCRIPTION

Referring to the Figures and more particularly to FIG. 1A, there is illustrated a stretchable bandage tape 10 in accordance with the present disclosure, for bandaging anatomical body parts (e.g., human beings and non-human animals as well). The tape 10 may be a monolithic and/or a continuous tape, made of silicone or silicone rubber. Silicone rubber is an elastomer (rubber-like material) composed of silicone and/or a polymer containing silicon together with carbon, hydrogen, and oxygen. Silicone rubbers are often one- or two-part polymers, and may contain fillers to improve properties. For the purpose of the present disclosure the word silicone will imply a material containing silicon and/or any mixture of silicone rubber. The present disclosure is based on the properties of silicone and silicone-rubber materials to stretch and bond to itself. The present disclosure also covers non-continuous sectioned portions of the self-fusing silicone and silicone-rubber. The sections of the tape 10 may be provided on a backing strip as described below (support liner, backing substrate) or on any specially coated surface to prevent the tape from sticking to itself. Furthermore, the sections of the tape 10 can be provided without a backing strip as a sized portion individually packaged. Such a package can contain more than one size of section of the tape 10 separated with a barrier to prevent sticking to each other or treated with a substance to prevent agglomeration. The tape 10 can be provided in any format including but not limited to individual piece, sheet, stripe, roll, fanfold (with pre-perforated tear lines 10A in FIG. 3, transversely positioned along the tape 10) or other. One essential property of the silicone is that it adheres to itself. When two layers of the tape 10 are overlaid, with a slight pressure being applied, the overlaid layers of the tape 10 fuse and become integral. The adhesion therebetween is dry, in that no adhesive of any sort needs to be applied to cause the adhesion. By having the tape in a monolithic configuration, both sides of the tape X are silicone or silicone rubber, and therefore self-adhering (a.k.a., self-fusing, self-amalgamating). This may allow the tape 10 to be used to surround a body part as a bandage. Moreover, the free ends of a same side may be connected to each other to create a flag. Although described as monolithic, the tape 10 may have a composite construction, or may have a core covered by the silicone or silicone rubber.

The bandage tape 10 may come in substantial length, for a limited thickness and width. As explained below, the thickness is selected such that the tape 10 has some elasticity when manually pulled or stretched. According to an embodiment, a suitable thickness range is between 0.5 mil-120.0 mil. With such thickness, the tape 10 may be ripped manually or easily cut off as in FIG. 1A, for instance when a sufficient length of tape 10 has been unrolled.

The width may depend on the application with which the bandage tape 10 is used, and more particularly the size of the wound or dressing. In a particular use to support dressing as explained after, the width of tape 10 may be equal or superior than that to the dressing width, although it may also be inferior as shown herein. According to an embodiment, the width may range between 0.25″ to 2.5″. It is also contemplated to use the tape 10 to apply to a larger area of a body, in which case the width can be between 2″ and 6″. These dimensional ranges are provided as an example only, and should be interpreted non-limitatively as convenient and practical dimensions for the contemplated use. Wrapping of larger objects, injured location or body or dressings is possible by using multiple overlapping layers of the bandage tape 10. Furthermore, a single piece of the bandage tape 10 or separate pieces of the tape 10 fused together may be used. Furthermore, the bandage tape 10 of sufficient thickness may be used to restrain movement of limbs and/or secure a body, for example on a stretcher.

As shown in FIG. 1A, the bandage tape 10 may come as a roll. In such arrangement, a backing strip 12 may be applied to one of the surfaces of the bandage tape 10, to protect the tape 10 from sticking to itself with its self-adhering properties. An applicator device may be used if desired to facilitate the dispensing and separation of a strip of the tape 10 with backing layer 12 from the roll. According to an embodiment, the length should be sufficient to reach the other end of the material upon stretching.

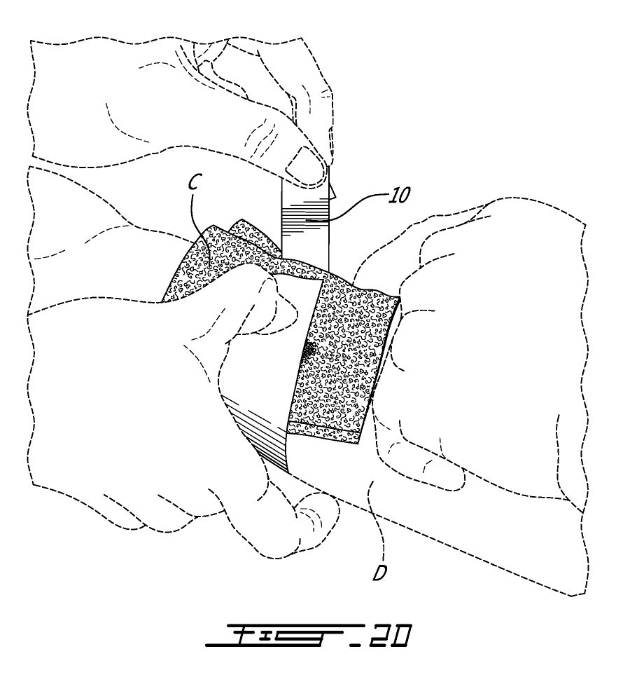

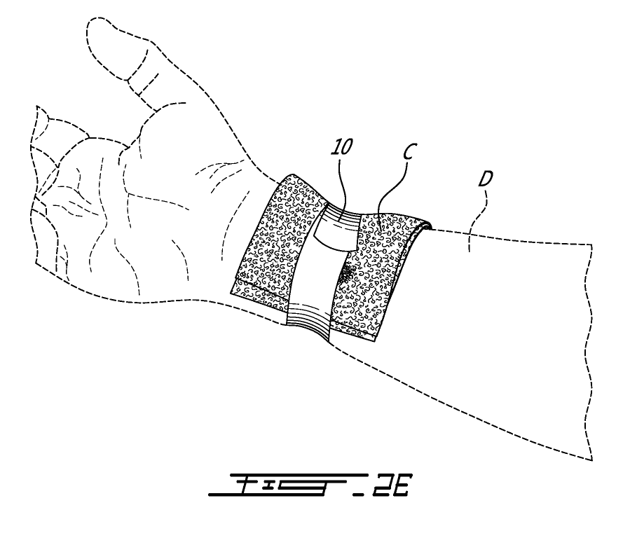

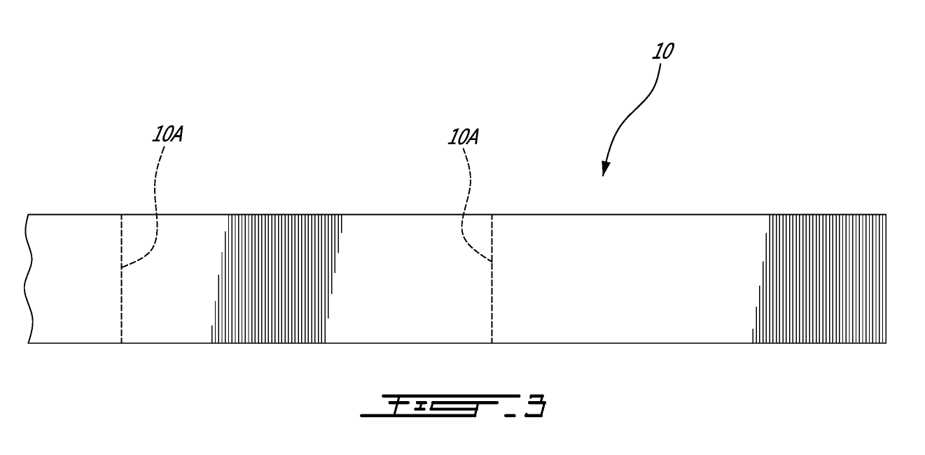

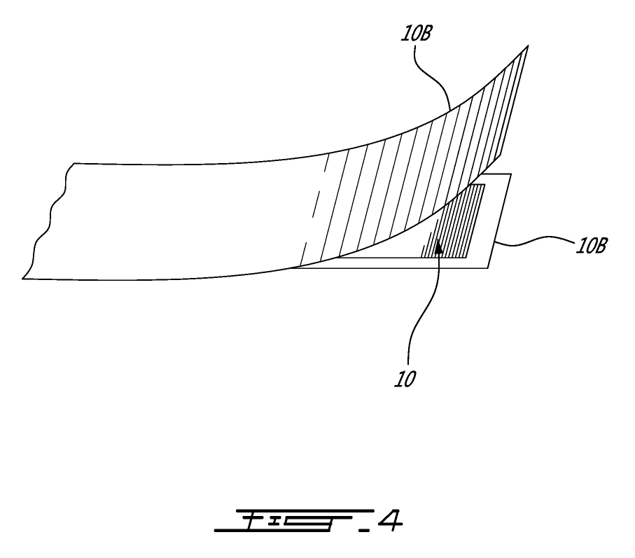

When a strip of the bandage tape 10 of suitable length has been obtained, the backing 12 may be removed from the tape 10 as in FIG. 1B. The tape 10 may come in pre-cut strips, or may be cut off or ripped from the roll of FIG. 1A. As another example, the tape 10 may be ripped from the roll when overlaying it onto itself to form the bandage. A first end of the tape 10 is firstly brought toward a wound A on the body portion, such as finger B as an example, as in FIG. 10. The tape 10, being sterilized, may be applied directly onto the wound A, to cover it, as in FIG. 10, by either one of its ends. Before the free end of the tape 10 is overlaid onto the applied end of the tape 10, the user exerts some pull on the free end of the tape 10 to stretch it. Therefore, when the tape 10 is overlaid on itself, as in FIG. 1E, it is in a stretched condition, and retained by its elasticity to the finger B. The tape 10 creates a non-penetrable barrier to water and can protect the injured area against contamination. The stretching adds elasticity to the formed bandage, by which it biases onto the finger B. The fusion after stretching and inherent coefficient of friction of silicone ensure that the formed bandage of tape 10 remains in position on the body, instead of sliding off. It is observed that a first side of the strip of tape 10 is overlaid onto a second side of the strip of tape 10: as the tape 10 is of monolithic construction, both sides have the self-fusing properties. Therefore, it is non-directional and reversible, such that the first surface at a first end of the strip of bandage tape 10 self-adheres to the second surface at a second end by overlaying, or the second surface at the first end self-adheres to the first surface at the second end by overlaying. The fusion may be permanent and may not be undone without ripping it apart. A less permanent or removable fusion may possibly be obtained depending on the chemical formulation and concentration of polymers and/or additives in the bandage tape 10. Because of the elasticity and permanent bond, the user must take the necessary precautions not to make the wrapping too tight which may cause stoppage or decrease in blood circulation.

Other uses or sequences may also apply. Referring to FIGS. 2A to 2E, the bandage tape 10 is used to secure a dressing C (e.g., gauze) to a limb D, such as the forearm as in FIG. 2A. Again, the tape 10 may come in pre-cut strips, or may be cut off or ripped from the roll, as in FIG. 2B. A first end of the tape 10 is applied directly onto the dressing C. Before the free end of the tape 10 is overlaid onto the applied end of the tape 10, the user exerts some pull on the free end of the tape 10 to stretch it as in FIG. 2D. Therefore, when the tape 10 is overlaid on itself, as in FIG. 2E, it is in a stretched condition, and attached by its elasticity to the forearm D. Although shown as only covering part of the dressing C, a larger width of the bandage 10 may be used to completely cover the dressing C and hence isolate it from the surroundings, for example forming a waterproof barrier. The bandage 10 of smaller width may also be wound spirally to completely cover the dressing C if desired.

If desired, the user may inscribe information on the tape 10, whether it be before applying it as a bandage or after it has been applied. Indeed, the material of the tape 10 will retain marker ink thereon. Any form of printer may be used to print information, or markers such as permanent ink markers.

If desired, the silicone bandage tape 10 can be provided in different colors for color coding according to medical alert system or medical nomenclature. Similarly, the color coding can be used in veterinary and related use. Other color-coding, identification methods, symbology or any other information can be printed on the silicone bandage tape 10 depending on the end-use application. The bandage tape 10 may be commercially printed if desired.

The silicone or silicone containing material of the bandage tape 10 gives it the capability of being applied over a surface which is wet in rain or snow conditions, mud, sand or dirt as a result of an accident or an environmental catastrophe, or disinfectant-wet or medication-wet surface, oily surface or when any type of ointment has been applied and create a hermetic seal to protect and maintain a sterile environment underneath the formed seal. The silicone and silicone-rubber bandage tape 10 is not affected by alcohol or other chemicals, solutions and ointments as it bonds to itself and remains bonded even when soaked in alcohol. This has useful implications for sterility protection Furthermore, since the material of the bandage tape 10 can withstand high temperatures (e.g., up to 260° C.), autoclave sterilization of a packaged tape and/or sized sections thereof can allow its use in surgical applications and in clean room applications. Furthermore, the silicone bandage tape 10 can be sterilized using gamma radiation, with sterilized sections of the silicone bandage tape 10 provided in individual sterile packages, as 10B in FIG. 4. The continuous tape in its entirety may also be provided in a sterile packaging. The bandage tape 10 may be provided in the form of an individually packaged portion in a sterile packaging or a plurality of individually packaged portions in the form of strips attached to each other with or without perforations (perforations in the form of a tear line might be used for easy separation of an individual package); or a plurality of individually packaged portions in a box or enclosure. Other types of packaging can be used. Other methods of sterilization of the bandage tape 10 are also contemplated, such as using ethylene oxide.

When the bandage tape 10 is in individually sized packages, it may have a dressing (such as gauze, cotton, etc.) attached to it via a glue, adhesive, or non-adhesive fusion allowing the wound dressing to remain fixed to the bandage tape 10. In such a case, the integral dressing would be aligned and applied onto the wound area or the skin and the dressing-free ends of the bandage tape 10 would be used for stretching and fusing with each other to hold the bandage tape 10 in place.

The bandage tape 10 may solve challenges: 1) it can secure a dressing on an injured area; 2) it can be applied directly to skin and assist in stopping a bleeding by creating a pressure; 3) it can be applied to wet and oily surfaces which may be present when there is an injury and the area is covered with blood and body fluids, sprayed with a disinfectant or with any ointment applied; 4) it will not stick to skin or to hair; 5) it can withstand high temperatures used in steam sterilization in autoclaves; 6) it can be provided in sterile packaging exposed to gamma irradiation; and/or 7) it can be used for other types of sterilization, such as ethylene oxide.

The present disclosure also describes the method of using the silicone tape for medical and veterinary applications comprising the following steps: a) cutting the desired portion of the bandage tape 10; b) removing the bandage tape 10 from the backing strip 12 if present; c) stretching the bandage 10 tape to a desired length; d) applying the bandage tape 10 to a dressing (or any equivalent) and/or directly to skin; e) bonding the tape 10 to itself. Although shown as contemplated sequence a)b)c)d)e), the steps can be done in any appropriate sequence, for example: b)a)c)d)e); b)c)d)a)e). Stretching described in step “c” is possible but not necessary and could be omitted. The step “a” may be a manually ripping off or tearing off instead of cutting with a sharp instrument. For an individually packaged portion, the tape 10 might not contain a backing strip and in this case the step of removing the tape 10 from the backing strip would be omitted.

The present disclosure also describes the method of using the sized portion of the bandage tape 10 for medical and veterinary applications comprising the following steps: f) opening the packaging to expose a section of the bandage tape 10; g) removing the backing strip 12 (if present) from the bandage tape 10; h) stretching the bandage tape 10 to a desired length; i) applying the bandage tape 10 to a dressing and/or directly to skin j); bonding the tape 10 to itself. Stretching described in step “h” is possible but not necessary and could be omitted.

The present disclosure also describes the method of using the sized portion of the bandage tape 10 without backing strip 12 in which it comprises the following steps: k) opening the packaging; l) stretching the tape 10 to desired length m) applying the tape 10 to a bandage and/or directly to skin n) bonding the tape to itself. Stretching described in step “I” is possible but not necessary and could be omitted.

The silicone bandage tape 10 can be provided as part of a kit comprising a manual dispenser or an automatic dispenser for cutting the desired length of the bandage tape 10. The kit might comprise any instrument capable of cutting the bandage tape 10.

The bandage tape 10 may have some or all of the following properties: 1) it is stretchable, for example up to 300% or more and hence will comply to movements of a body portion when applied to it; 2) it does not contain any surface layer of adhesive other than its self-adhering property; 3) it can be handled by naked hands and it will not stick to a human skin but it can adhere to itself; 4) once it is wrapped onto itself for example by stretching and wrapping around an object, or wrapping a splice, it creates an integral bond and tight seal that cannot be unwrapped without force after a period of time; 5) it can be applied to surfaces such as body extremities or limbs which are wet due to bleeding, body fluids, environmental conditions, applied liquids and ointments; 6) it can withstand various environmental conditions and physical friction; 7) it can be applied over clothing or a naked skin; 8) it can withstand a relatively wide temperature range from cold to warm while not losing its fusing and elastic properties; and/or 9) it is waterproof and weatherproof. Because of its elasticity, the bandage tape 10 may be used as a tourniquet to limit or stop venom, substance or infection transfer from the area of a wound including bites from reptiles, insects or other creatures or animals.

The bandage tape 10 may incorporate (e.g., embed) a wireless device such as GPS, cellular phone, Radio Frequency Identification (RFID), Near Field Communication (NFC) tag technologies or any tag or device that comprises wireless communication capability. This technology allows the reading of the information without a physical contact with the wireless tag. According to an embodiment, the wireless device is positioned onto a first layer of the tape 10, with a second layer then overlaid on the first layer of tape 10, such that the wireless device is sandwiched between two layers of the tape 10, and thus fully encapsulated by the tape 10, and therefore attached to the patient. These wireless devices supported by the tape 10 may contain valuable information about the location of the patient, information about the medical condition of the patient, blood type, type of allergy, or any other information related to patient. The bandage tape 10 may also incorporate a wire, a cable, wired electronics, a memory chip, a connector or an electronic device or electrical connection for being connected to a medical device.

The present disclosure also covers when the silicone or silicone-rubber bandage tape 10 is applied on a body part over a clothing or dress such as a shirt, pullover, jacket, suit, skirt, underwear or any outer clothing designed to be worn. Artificial body parts are covered by the present disclosure. The bandage tape 10 with suitable thickness and width may provide the suitable strength to be used for movement restraint, for example in law enforcement and in other medical and non-medical facilities for restraining an individual or animal for example by tying-up extremities to each other or tying-up extremities to an object with the bandage tape 10.

These wireless devices supported by the tape 10 may contain valuable information about the location of the patient, hospital or clinic name, doctor's name, name of the patient, address of the patient, contact information, information about the medical condition of the patient, name of a disease or condition, name of a bacteria, virus, protozoa, parasite, warms, injury type, cardiogram, symptom, blood type, type of allergy, or any other information related to patient, cause of a medical condition and description of a medical condition.

1. A bandage tape comprising a strip body having a first surface and a second surface, the first surface and the second surface being made of one of a silicone and a silicone rubber, a thickness between the first surface and the second surface ranging between 0.5 mil-120.0 mil, the strip body configured for the first surface at a first end to self-adhere to the second surface at a second end by overlaying in a silicone-to-silicone bond, the strip body further configured for the second surface at the first end to self-adhere to the first surface at the second end by overlaying in a silicone-to-silicone bond.

2. The bandage tape of claim 1, wherein the strip body is a monolithic body of silicone or silicon rubber.

3. The bandage tape of claim 1, further comprising printing on at least one of the first surface and of the second surface.

4. The bandage tape of claim 1, wherein the strip body is non-continuous and divided into a section sized for a contemplated used.

5. The bandage tape of claim 4, wherein the non-continuous strip body is in an individual package in a sterile condition.

6. The bandage tape of claim 1, wherein the strip body is in a roll, and further comprising a backing layer placed between the first surface and the second surface of the bandage tape in the roll.

7. The bandage tape of claim 6, further comprising pre-perforated tear lines transversely positioned and spaced apart along the strip body.

8. The bandage tape of claim 1, wherein the strip body has a width ranging between 0.25″ and 6.0″.

9. A method for bandaging an anatomical body part comprising:
obtaining a bandage tape comprising a strip body having a first surface and a second surface, the first surface and the second surface being made of one of a silicone and a silicone rubber, a thickness between the first surface and the second surface ranging between 0.5 mil-120.0 mil;
placing a first end of the strip body against the anatomical body part to be bandaged;
wrapping and stretching the strip body around the anatomical body part; and
forming a silicone-to-silicone bond by overlaying the second surface at a second end onto the first surface at the first end, whereby the strip body self-adheres to itself while stretched around the anatomical body part.

10. The method according to claim 9, wherein forming the silicone-to-silicone bond by overlaying the second surface at a second end onto the first surface at the first end comprises forming the silicone-to-silicone bond while the strip body is over a wound on the anatomical body part, whereby the strip body self-adheres to itself while stretched to cover said wound of the anatomical body part.

11. The method according to claim 10, wherein forming the silicone-to-silicone bond while the strip body is over a wound on the anatomical body part comprises forming the silicone-to-silicone bond while the strip body is directly in contact with said wound.

12. The method according to claim 10, further comprising placing a dressing on the wound, and wherein forming the silicone-to-silicone bond includes forming the silicone-to-silicone bond such that the dressing is between the wound and the strip body.

13. (canceled)

14. The method according to claim 9, further comprising applying at least one of a disinfectant and ointment to the anatomical body part prior to bandaging the anatomical body part, whereby the bandage tape is directly in contact with the at least one of a disinfectant and ointment.

15. The method according to claim 9, further comprising applying medication to the anatomical body part prior to bandaging the anatomical body part, whereby the medication is held captive and in contact with the anatomical body part.

16. The method according to claim 9, wherein stretching the strip body around the anatomical body part comprises stretching the strip body by at least 50%.

17. The method according to claim 9, wherein obtaining a bandage tape comprises unrolling a length of the bandage tape from a roll, sectioning the bandage tape to define the strip body, and removing a backing layer from the strip body.

18. The method according to claim 9, wherein obtaining a bandage tape comprises removing the strip body from an individual package.

19. The method according to claim 9, further comprising placing a wireless device between the strip body and the anatomical body part, whereby the wireless device is maintained captive against the anatomical body part.

20. The method according to claim 9, further comprising placing a medical device interacting with the anatomical body part between the strip body and the anatomical body part, whereby the medical device is maintained captive against the anatomical body part.

21.-22. (canceled)

23. The method according to claim 9, wherein forming the silicone-to-silicone bond by overlaying the second surface at a second end onto the first surface at the first end comprises forming a tourniquet to reduce blood flow on the anatomical body part.

24.-39. (canceled)
